(19) United States
(12) Patent Application Publication
(10) Pub. No.: US 2013/0289349 A1
(43) Pub. Date: Oct. 31, 2013

(54) Small Diameter Endoscope for Pain Management

(71) Applicants: John Costin, Lorain, OH (US); Nagy Mehail, Cleveland, OH (US); Elliott Reed, Cleveland, OH (US); Subba Shankar, Cleveland, OH (US)

(72) Inventors: John Costin, Lorain, OH (US); Nagy Mehail, Cleveland, OH (US); Elliott Reed, Cleveland, OH (US); Subba Shankar, Cleveland, OH (US)

(73) Assignee: CLEAR IMAGE TECHNOLOGY, LLC

(21) Appl. No.: 13/855,527

(22) Filed: Apr. 2, 2013

Related U.S. Application Data
(60) Provisional application No. 61/620,519, filed on Apr. 5, 2012.

(51) Int. Cl. A61B 1/015

(52) U.S. Cl. CPC A61B 1/015
USPC 600/109; 600/153; 600/156

(57) ABSTRACT

A small diameter, multi-channel endoscope for pain management in a subject includes a control handle and a flexible, elongated body functionally connected to the control handle. The body includes a distal end for positioning in or about a target structure. The body also includes a working channel that extends longitudinally through the body. The working channel includes a controllable deployment mechanism configured for selective application of at least one target substance to the target structure. Additionally, the body includes an image channel extending longitudinally through the body. The image channel includes a visualization system for conveying an image from the distal end of the body to a user.

This application claims priority from Application No. 61/620,519, filed Apr. 5, 2012, the entire contents of which are herewith incorporated by reference.

### BACKGROUND

Advancements in continual and delayed release drugs for pain relief can overcome the short duration of the traditional therapeutic. A drug may be implanted into a body cavity, e.g., the epidural space.

Maximum effectivity is obtained when the drug is delivered into in close proximity to a target nerve. However, the conventional system of this type uses fluoroscopy, and in so doing, there is no ability to visualize and confirm the correct placement of the drug. For example, conventional spine interventional pain procedures utilize x-ray fluoroscopy to guide placement of a needle into the epidural space for delivery of pain treatment therapy. Using fluoroscopy, however, there is neither the ability to visualize and confirm correct placement of a pain treatment therapy, nor the ability to reposition or manipulate the therapy (if necessary).

### SUMMARY

The present invention relates generally to a device to a small diameter, multi-channel endoscope with imaging and substance placing capabilities.

One aspect of the present invention includes a small diameter, multi-channel endoscope for implanting, visualizing, positioning and repositioning a drug or other substance in a body cavity of a subject.

In embodiments, the endoscope has a control portion e.g., with a handle, and an elongated body functionally connected to the control handle. The body includes multiple channels extending through the body including a controllable deployment mechanism configured for selective application of a target substance to a target location in the human body. Additionally, the body includes an image channel extending longitudinally through the body that allows obtaining an image from the distal end of the body to a user. In embodiments, other channels are provided for cleaning the distal end and/or cleaning the media to clarify the image, and for illuminating the target area in the body.

In one aspect, this can be used for pain management in a subject by implanting a drug to precisely the right location.

In one embodiment, the drug is a capsule or pellet.

In one embodiment, the item that is implanted is capable and suspectable of being repositioned.

In one embodiment, the endoscope can be used to view the anatomical environment and a user, e.g., a physician, can determine if previous actions had altered the anatomy.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view showing an endoscope constructed in accordance with one aspect of the present invention;

FIG. 2 is a cross-sectional view taken along Line 3-3 in FIG. 1;

FIG. 3 is a magnified, partial cross-sectional view taken along a longitudinal axis of the endoscope in FIG. 1;

FIG. 4 is a perspective view showing a small diameter, multi-channel endoscope constructed in accordance with another embodiment of the present invention;

FIG. 5 is a cross-sectional view taken along Line 7-7 in FIG. 4;

FIG. 6 is a magnified, partial cross-sectional view taken along a longitudinal axis of the endoscope in FIG. 4;

FIG. 7 is a magnified, perspective view showing a therapeutic pellet being deployed from the endoscope in FIG. 4; and

FIG. 8 is a magnified, perspective view showing the internal construction of the endoscope in FIG. 7.

### DETAILED DESCRIPTION

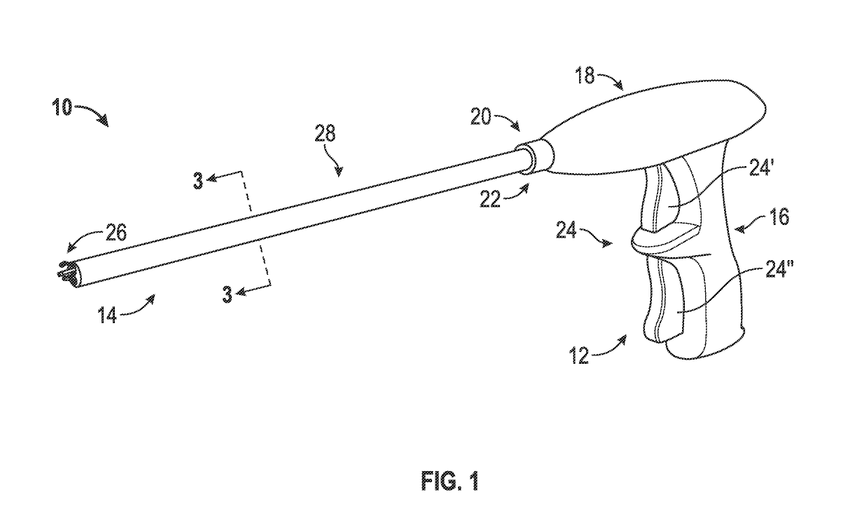

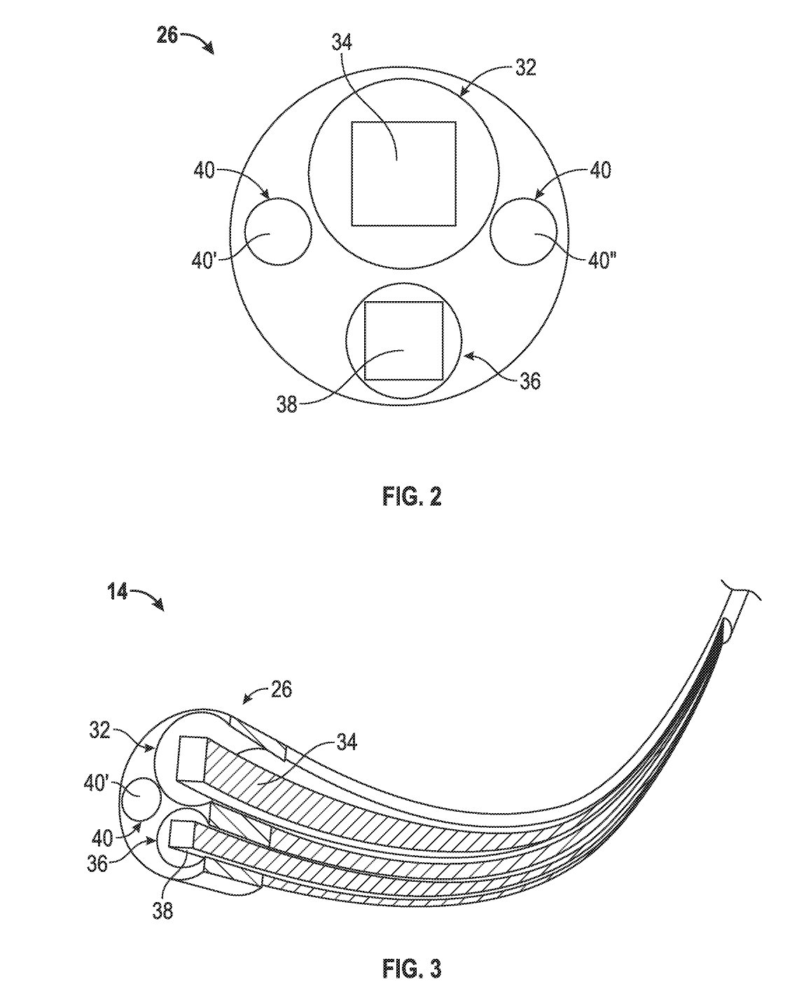

FIG. 1 illustrates a multi-channel endoscope 10 according to an embodiment. As explained herein, the endoscope 10 includes plural different channels, each of which that allow different operations. A cross section across the line 3-3 is shown in FIG. 2, showing the different channels that extend through the elongated tube of the endoscope. As explained herein, one of the channels can be used to implant a drug into a patient. Other channels can be used to move the drug and/or to visualize its location.

The endoscope 10 is in a housing with a control handle 12 that is functionally connected to a flexible, elongated body 14. The entire endoscope 10 can be disposable and configured for single-use applications. Alternatively, only a portion of the endoscope 10 can be disposable and configured for single-use applications. For example, only the body 14 of the endoscope 10 may be disposable and configured for single-use applications, and a new body can be attached to the handle.

The control handle 12 has an ergonomic configuration to provide grip and maneuverability to the endoscope 10. The control handle 12 includes a lower portion 16 that is integrally formed with an upper portion 18. The upper portion 18 includes a connection part 20 holds the tubular endoscope body 14. As described in more detail below, the lower portion 16 can include one or more buttons 24, 24′ configured to control certain features or components of the endoscope 10. All or only a portion of the control handle 12 can be made of a rigid or semi-rigid medical grade metal or metal alloy, such as stainless steel, medical grade plastics, polymers, or the like. It will be appreciated that the control handle 12 can include various features to provide grip and tactile maneuverability, such as circumferential ridges or a cross-hatched precut pattern material forming the control handle.

The body 14 of the endoscope 10 has an elongated, tubular configuration. A proximal end 22 is connected to the handle portion 12, and in one embodiment removable therefrom. The opposite end of the body 14 is a distal end 26. An intermediate portion 28 that extends between the proximal and distal ends.

The body 14 has an approximately cylindrical or tubular configuration; however, it will be appreciated that the body can have other geometric configurations (e.g., a square or ovoid-shaped cross-sectional profile). The distal end 26 of the body 14 is configured for positioning in or about a target structure of a human body. The body 14 can be semi-rigid or, alternatively, all or only a portion of the body can be comprised of one or more materials such that the body can articulate. The outer diameter of the body is preferably such that the body can fit within a cannula to be inserted into a human body.

FIG. 2 shows a cross section across the line 3-3 in FIG. 1 and FIG. 3 shows a side view along a cross section. The body 14 comprises a plurality of channels 34, 38, 40′ and 40″, that longitudinally extend between the proximal and distal ends of the body 14. Each of the channels is shown a circular cross-sectional profile, it will be appreciated that some or all of the channels can have other cross-sectional profiles, such as square or ovoid.

The working channel 32 may be the largest diameter of the channels. This channel can include a controllable deployment mechanism 34 that is configured for selective application of at least one target substance to a target structure (described in more detail below). In one embodiment, the target substance is a small pellet including a drug, to be applied to a very specific location, e.g., near a nerve. Any or all of these can be included as part of the “substance” to be delivered.

The deployment mechanism 34 can depend on the type of target substance to be delivered to the target structure, the location of the target structure, and the overall dimensions of the endoscope 10. The movement of the deployment mechanism 34 can be controlled by a user (e.g., a physician) depressing the buttons 24′ and 24″. For example, the control handle 12 can include a first button 24′ that, when actuated, causes the deployment mechanism 34 to dispense or apply at least one target substance in or about a target structure. The first button 24′ can be further actuated to allow a user to selectively control the deployment mechanism 34 and physically manipulate the target substance. The control handle 12 can additionally include a second button 24″ that, when actuated, selectively loads another target substance into the deployment mechanism 34. Buttons and controls can also be provided for moving the mechanism 34 back and forth, grabbing and un grabbing the substance, illuminating, washing the camera lens, and other parts of the control.

The body 14 of the endoscope 10 further includes an image channel 36 which includes a visualization system 38 for conveying an image from the distal end 26 of the body 14 to a user. The visualization system 38 can include any one or combination of imaging components that enable direct visualization of biological tissue during use of the endoscope 10. For example, the visualization system 38 can include an optical cable configured to transmit illumination from a proximal end to a distal end thereof while transmitting the image from the distal end to a user. For example, system can include a CMOS or CCD camera module at either end, and illumination can be provided by an LED illuminating a fiber-optic or a light-pipe made of an optically clear polymer (e.g., acrylic, polycarbonate). By locating the camera and optics at the side of the control device 18, these can be reused even if the body is disposable.

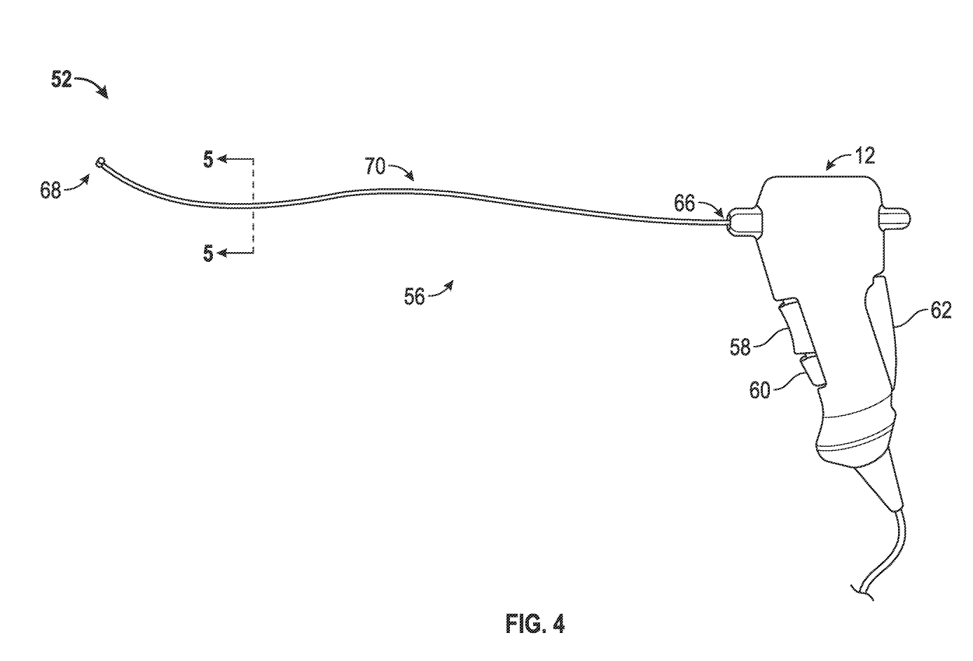

The body 14 of the endoscope 10 can additionally include fluid channels 40′ and 40″. A first fluid channel 40′ that is oppositely disposed from a second fluid channel 40″ (FIGS. 3-4). One of the fluid channels 40′ can dispense a fluid (e.g., a tumescent fluid) during operation of the endoscope 10, and the other can remove fluid in the area. In this case, the endoscope 10 can include a fluid pump and suction apparatus that is communication with one or both of the fluid channels 40′ and/or 40″. Alternatively, one or both of the fluid channels 40′ and/or 40″ can be configured to provide suction at the distal end 26 of the body 14. In this case, the endoscope 10 can include a vacuum pump or source of negative pressure that is in communication with one or both of the fluid channels 40′ and/or 40″. Preferably, the channel 40′ is delivering fluid, while the channel 40″ is vacuuming fluid. This keeps the area clean, making a better image.

In operation of one embodiment, the deployment mechanism 34 is pre-loaded with a desired amount of one or a combination of target substances, e.g., drugs. The particular choice of target substance will depend upon one or more factors, including the specific ailment being treated. Alternatively, to treat sciatica or similar nerve disorder, a pharmaceutical composition comprising an analgesic may be chosen for administration to a sciatic nerve. One skilled in the art will appreciate how to determine the appropriate target substance.

The particular construction of the deployment mechanism 34 will depend, at least in part, on the particular type of target substance and target structure. For nerve stimulation, for instance, the deployment mechanism 34 can comprise an electrical lead-like apparatus configured to selectively apply electrical energy to a target nerve structure. Alternatively, for delivery of a pharmaceutical composition to a target structure, the deployment mechanism 34 can be configured similar to a syringe or pump, which is adapted for selectively applying a desired volume of the pharmaceutical composition to the target structure.

After the endoscope 10 has the one or more target substances, either preloaded or placed otherwise to be delivered by the body 14 is surgically advanced to a target structure under direct visualization. Depending upon the anatomical location and nature of the target structure, the distal end 26 of the endoscope 10 can be positioned in direct contact with the target structure or, alternatively, in effective proximity to the target structure. By “effective proximity”, it is meant that the distal end 26 of the body 14 can be positioned at a distance from the target structure sufficient to deliver a therapeutically effective amount of at least one target substance to the target structure. Advantageously, advancement of the distal end 26 under direct visualization removes the need for fluoroscopy-based procedures and allows the user to accurately and precisely position the distal end in or about the target structure.

The user operates the control handle 12 to actuate the deployment mechanism 34 and thereby dispense the at least one target substance at the target site. For example, a user can actuate the first button 24′ of the control handle 12, which in turn causes the deployment mechanism 34 to dispense the at least one target substance at the target site. The deployment mechanism 34 can be actuated only a single time to dispense the at least one target substance or, alternatively, the deployment mechanism can be repeatedly actuated to periodically dispense multiple doses or applications of the at least one target substance.

During and/or after application of the at least one target substance, efficacy of the treatment can be assessed by asking the subject to report on whether their pain level have improved or by imaging the area of delivery to determine if the area is where the manufacturer has recommended locating the drug. If the level of pain has not improved, the drug can be moved or re-applied until the subject reports a decrease or resolution in the pain level.

For example, the endoscope 10 can be re-loaded with the same or different target substance and then reapplied to the same or different target structure until the subject reports an improvement in the level of pain. In some instances, physical adjustment or repositioning of the at least one target substance (e.g., by actuating the second button 24″) may be required. In this case, the user can actuate the first button 24′ of the control handle 12 to selectively control the deployment mechanism 34 and thereby reposition the at least one target substance. Advantageously, selective placement and manipulation of a single target substance increase precision and efficacy of the embodiment. If the subject reports a decrease or resolution of pain symptoms, the endoscope 10 can be removed from the subject and the procedure completed.

FIG. 4 illustrates an embodiment, having endoscope 52 for drug or substance delivery, e.g. for pain management. The endoscope 52 shown in FIG. 4 is shaped, dimensioned, and configured to deposit one or more therapeutic pellets 53 (FIG. 6) configured for long-term pain management at or about a target structure (e.g., an intervertebral disc or a peripheral nerve) under direct visualization. The depositing is done through one channel, and lighting and visualization through another channel. Therapeutic pellets 53 deliverable by the endoscope 52 can comprise time-release, biocompatible and/or biodegradable bead-like structures that include one or more target substances. Advantageously, the endoscope 52 provides a direct visualization pain therapy delivery system that not only obviates the need for fluoroscopy-assisted procedures, but also allows a user the ability to reposition a misplaced therapeutic pellet 53 (or pellets).

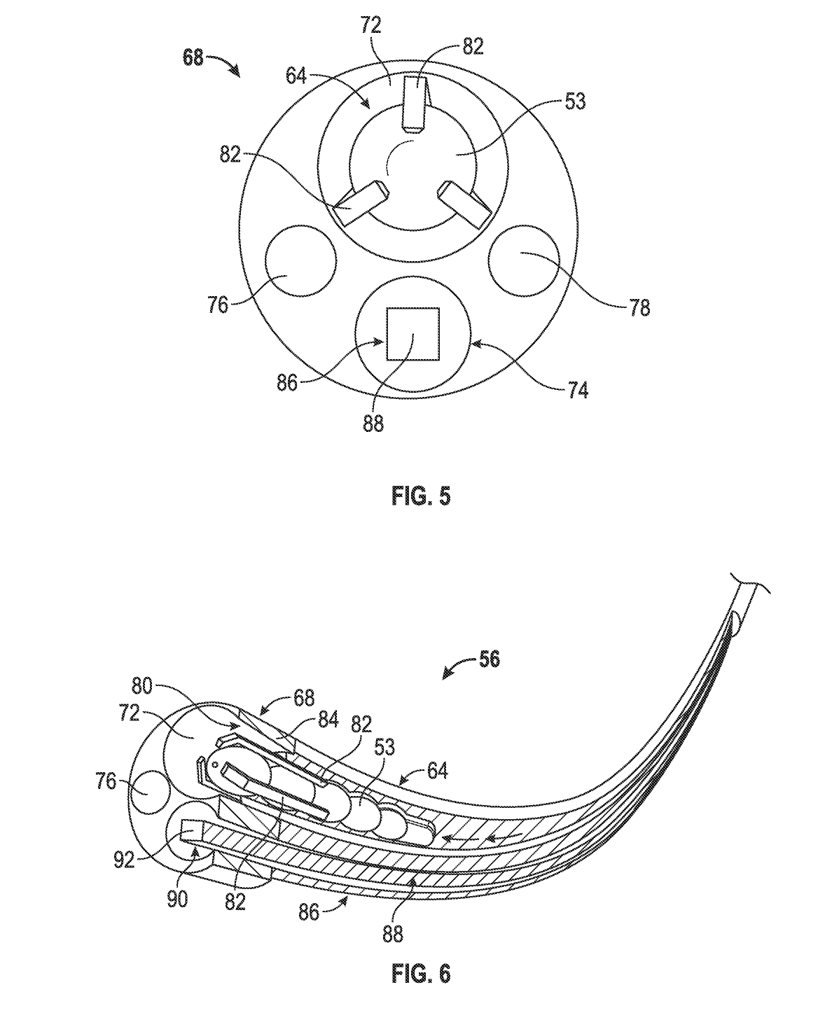

The endoscope 52 has a control handle 54 that is functionally connected to a flexible, elongated body 56. The entire endoscope 52 can be disposable and configured for single-use applications. Alternatively, only a portion of the endoscope 52 can be disposable and configured for single-use applications. For example, only the body 56 of the endoscope 52 may be disposable and configured for single-use applications. The control handle 54 has an ergonomic configuration to provide grip and maneuverability to the endoscope 52. The control handle 54 includes first, second, and third buttons 58, 60, and 62 configured to control certain features or components of the endoscope 52, such as a deployment mechanism 64 (FIG. 5). As described in more detail below, the first button 58 can be actuated to deliver and reposition at least one therapeutic pellet 53 to a target structure, the second button 60 can be actuated to re-load a therapeutic pellet (or pellets) in the deployment mechanism 64, and the third button 62 can be actuated to articulate the body 56 of the endoscope 52. All or only a portion of the control handle 54 can be made of a rigid or semi-rigid medical grade metal or metal alloy, such as stainless steel, medical grade plastics, polymers, or the like.

The body 56 of the endoscope 52 has an elongated, flexible configuration and includes a proximal end 66, a distal end 68, and an intermediate portion 70 that extends between the proximal and distal ends. As shown in FIG. 5, the body 56 has an approximately cylindrical or tubular configuration; however, it will be appreciated that the body can have other geometric configurations (e.g., a square or ovoid-shaped cross-sectional profile). The body 56 is preferably as thin as possible so that it can fit through a cannula. The distal end 68 of the body 56 is configured for positioning in or about a target structure. All or only a portion of the body 56 can be comprised of one or more materials so that the body can articulate.

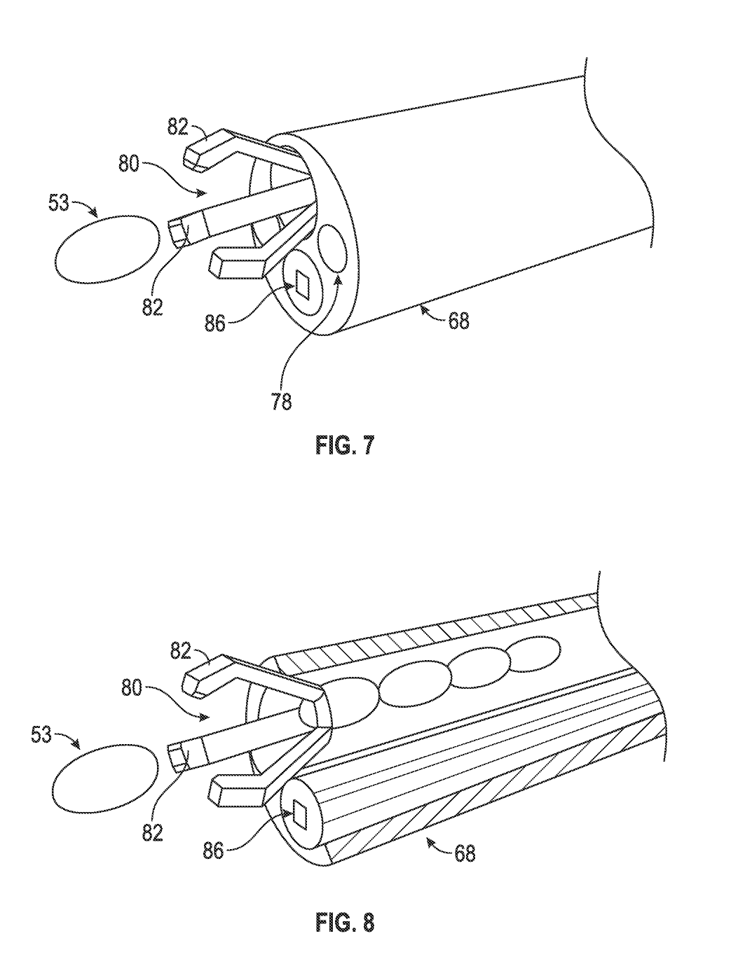

The body 56 (FIGS. 7-8) comprises a working channel 72, an image channel 74, and first and second fluid channels 76 and 78 that longitudinally extend between the proximal and distal ends 66 and 68 of the body. Although each of the channels 72, 74, 76, and 78 has a circular cross-sectional profile, it will be appreciated that some or all of the channels can have other cross-sectional profiles, such as square or ovoid.

The working channel 72 includes a controllable deployment mechanism 64 that is configured for selective application of at least one therapeutic pellet 53 (FIG. 8) to a target structure. As shown in FIGS. 7-8, the deployment mechanism 64 generally includes an elongate grasping arm 80 comprising a plurality of flexible grasping members 82. The grasping arm 80 is capable of transitioning between a non-deployed configuration (FIG. 6) and a deployed configuration (FIGS. 7-8). In the non-deployed configuration, the grasping members 82 are substantially coaxial with the body 56 of the endoscope 52, and collectively form a securing surface 84 configured to retain one or more therapeutic pellets 53. In the deployed configuration, the grasping members 82 flare radially outward to release the therapeutic pellet(s) 53. As described in more detail below, the deployed configuration also enables a user to selectively control the grasping arm 80 and reposition a therapeutic pellet 53 (or pellets) following application the pellet(s) in or about a target structure.

A proximal end (not shown) of the deployment mechanism 64 is functionally connected to each of the first, second, and third buttons 58, 60, and 62 of the control handle 54. As described in more detail below, a user can actuate each the first and second buttons 58 and 60 of the control handle 54 to selectively control operation of the deployment mechanism 64 and, thus, application, repositioning, and re-loading of the therapeutic pellet(s) 53. For example, a user can actuate the first button 58 to cause the deployment mechanism 64 to release a single therapeutic pellet 53 in or about a target structure. Alternatively, a user can actuate the first button 58 to cause the deployment mechanism 64 to release multiple therapeutic pellets 53 in or about a target structure.

The body 56 of the endoscope 52 further includes an image channel 74. The image channel 74 includes a visualization system 86 for conveying an image from the distal end 68 of the body 56 to a user. The visualization system 86 can include any one or combination of imaging components (not shown) that enable direct visualization of biological tissue during use of the endoscope 52. For example, the visualization system 86 can include an optical cable 88 configured to transmit illumination from a proximal end (not shown) to a distal end 90 thereof while transmitting the image from the distal end to a user. Illumination can be provided by one or a combination known lighting components, such as LEDs located at the distal end of the endoscope, fiber-optics, or a light-pipe made of an optically clear polymer (e.g., acrylic, polycarbonate). The image can be transmitted to the user in different forms, e.g., as different forms of energy. In the case where the camera is in the handle, the image itself, e.g., the photons, can be transmitted down the optical cable. If the camera is on the endoscope end, the electrical energy indicative of the converted image can be transmitted.

In one embodiment, the optical cable 88 can comprise a small diameter fiberscope having a small camera module 92 (e.g., CMOS or CCD) located at the distal end 90 thereof. The camera module 92 can be a single plane imaging sensor with imaging and control logic on the same chip. Alternatively, the imaging region may be placed on top of the control logic. In another example of the present invention, the optical cable 88 can be configured in an identical or similar manner as those commercially available from Clear Image Technologies, LLC (Elyria, Ohio).

As shown in FIGS. 5-6, the body 56 of the endoscope 52 additionally includes first and second fluid channels 76 and 78, each of which is configured to convey a fluid therethrough. The first fluid channel 76 and/or the second fluid channel 78 can be configured to dispense a fluid (e.g., a tumescent fluid) during operation of the endoscope 52 to assist in visualizing a target structure, such as a nerve. In this case, the endoscope 52 can include a fluid pump that is communication with the first fluid channel 76 and/or the second fluid channel 78. Alternatively, the first fluid channel 76 and/or the second fluid channel 78 can be configured to provide suction at the distal end 68 of the body 56. In this case, the endoscope 52 can include a vacuum pump (not shown) or source of negative pressure that is in communication with the first fluid channel 76 and/or the second fluid channel 78.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, other substances can be delivered, and other ways of delivering the substances can be used.

Many of the functions described herein can be carried out by a computer. The functions may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

The various illustrative logical blocks, modules, and circuits described in connection with the embodiments disclosed herein, may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, displayport, or any other form. This may include laptop or desktop computers, and may also include portable computers, including cell phones, tablets such as the IPAD™, and all other kinds of computers and computing platforms.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein.

The steps of a method or algorithm described in connection with the embodiments disclosed herein may be embodied directly in hardware, in a software module executed by a processor, using cloud computing, or in combinations. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of tangible storage medium that stores tangible, non transitory computer based instructions. An exemplary storage medium is coupled to the processor such that the processor can read information from, and write information to, the storage medium. In the alternative, the storage medium may be integral to the processor. The processor and the storage medium may reside in reconfigurable logic of any type.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

The memory storage can also be rotating magnetic hard disk drives, optical disk drives, or flash memory based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media. The computer readable media can be an article comprising a machine-readable non-transitory tangible medium embodying information indicative of instructions that when performed by one or more machines result in computer implemented operations comprising the actions described throughout this specification.

Operations as described herein can be carried out on or over a website. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Also, the inventor(s) intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The programs may be written in C, or Java, Brew or any other programming language. The programs may be resident on a storage medium, e.g., magnetic or optical, e.g. the computer hard drive, a removable disk or media such as a memory stick or SD media, or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An endoscope comprising:
a control handle,
an elongated body, connected to the control handle;
said body including multiple separated channels extending through from a first end near said control handle to a second end,
said multiple separated channels including a first channel that extends from the first end to the second end, and includes a controllable deployment mechanism that is controlled by a control on said control handle for selective application of a substance to a target structure adjacent the second end;
said multiple separated channels also including an image channel that extends from the first end to the second end, and includes a visualization system for conveying an image from said second end to a user.

2. The endoscope of claim 1, wherein said deployment mechanism is configured to physically manipulate the substance while in a human body, in an area adjacent the second end.

3. The endoscope of claim 1, wherein said body further comprises first and second fluid channels extending longitudinally through said body, the first fluid channel providing a cleaning fluid to the second end, and the second fluid channel removing fluid from an area of the second end.

4. The endoscope of claim 1, wherein said visualization system further comprises an optical cable configured to transmit illumination to the second end while transmitting the image from the second end.

5. The endoscope of claim 4, wherein said second end of said optical cable includes a CMOS or CCD camera module.

6. The endoscope of claim 1, wherein said substance is a pellet, and said deployment mechanism is a gripper which holds the pellet.

7. The endoscope of claim 3, wherein said first channel is larger in diameter than other channels.

8. An endoscope comprising:
a control handle,
an elongated body, connected to the control handle;
said body including
a first channel that extends from a first end close to the handle to a second end, and includes a controllable deployment mechanism that is controlled by a control on said control handle for selective application of a substance from the endoscope to a target structure adjacent the second end,
an image channel, smaller in diameter than the first channel, and that extends from the first end to the second end, and includes an optical system sending an optical view from the second end to the first end, and
first and second fluid channels extending longitudinally through said body, the first fluid channel providing a cleaning fluid, and the second fluid channel removing fluid from an area of said image channel.

9. The endoscope of claim 8, wherein said deployment mechanism is configured to physically manipulate the substance while in a human body, in an area adjacent the second end.

10. The endoscope of claim 8, wherein said image channel further comprises an optical cable configured to transmit illumination to the second end while transmitting the image from the second end.
